(19) United States
(12) Patent Application Publication
(10) Pub. No.: US 2013/0178949 A1
(43) Pub. Date: Jul. 11, 2013

(54) AIR IMPEDANCE ELECTROSPINNING FOR CONTROLLED POROSITY

(71) Applicants: Gary L. Bowlin, Mechanicsville, VA (US); Michael J. McClure, Richmond, VA (US); David G. Simpson, Mechanicsville, VA (US); Hu Yang, Mechanicsville, VA (US)

(72) Inventors: Gary L. Bowlin, Mechanicsville, VA (US); Michael J. McClure, Richmond, VA (US); David G. Simpson, Mechanicsville, VA (US); Hu Yang, Mechanicsville, VA (US)

(73) Assignee: Virginia Commonwealth University, Richmond, VA (US)

(21) Appl. No.: 13/811,286

(22) PCT Filed: Jun. 28, 2011
(86) PCT No.: PCT/US11/42144

§ 371 Date: Jan. 21, 2013

Related U.S. Application Data
(60) Provisional application No. 61/359,200, filed on Jun. 28, 2010.

(51) Int. Cl. A61F 2/02

(52) U.S. Cl. CPC A61F 2/02
USPC 623/23.72; 425/8; 264/6

(57) ABSTRACT

Electrospun materials are fabricated using air-flow impedance technology, which results in the production of scaffolds in which some regions are dense with low porosity and others regions are less dense and more porous. The dense regions provide structural support for the scaffold while the porous regions permit entry of cells and other materials into the scaffold, e.g. when used for tissue engineering.

### BACKGROUND OF THE INVENTION

1. Field of the Invention

The invention generally relates to electrospinning materials using a mandrel designed to provide air impedance during spinning operations so as to produce electrospun materials (e.g. tissue engineering scaffolds) which comprise regions of differing fiber densities and/or porosities. In particular, due to their fabrication using air-flow impedance technology, some regions of the electrospun materials are dense, exhibit low porosity and provide structural support for the material. Other regions are, by comparison, porous, permitting entry of cells (and other materials) into the scaffold, and the migration of cells along the fibers, resulting in accelerated penetration of cells into a scaffold and/or more uniform distribution of cells within the scaffold.

2. Background of the Invention

The goal of any tissue engineering approach is to develop scaffolds that are capable of functional regeneration. To duplicate all the essential intercellular reactions and promote native intracellular responses, the goal is to reproduce the structure and/or function of the native extracellular matrix (ECM). The ECM analogues, or scaffolds, should conform to a specific set of requirements [1-3]. In native tissues, the structural ECM proteins (50-300 nm) are one to two orders of magnitude smaller than the cell itself which allows the cell to be in direct contact with many ECM fibers and define its 3-D orientation. Thus, engineers have tried to replicate this fibrous structure to serve as a scaffold for cell seeding and tissue development. However, it has been difficult to establish an even 3-D distribution of cells in any scaffolding regardless of scaffold fabrication method or cell seeding technique.

Electrospinning represents a processing method to meet both the general material requirements as well as the potential size issues and has been described extensively in terms of the process [4-6] and its potential applications in tissue engineering [7, 8]. The major limitation of electrospinning is the inability to control pore size and overall porosity of the scaffolds due the random deposition and packing of fibers to form a non-woven fibrous structure. This fine pore structure limits the ability to seed the scaffold, more often than not, allowing only cell seeding of the surface and relying on subsequent cell migration (restricted by the fine pore structures) into the structure. Conventional electrospun scaffolds deposit as layers of fibers. Few if any fibers are oriented perpendicular to the horizontal axis of these fiber arrays. When cells are seeded onto an electrospun scaffold they tend to spread over the surface of the structure and do not penetrate efficiently across the fibers into the deeper layers of the structure. They tend to spread along the fibers rather than across the fibers. This can be attributed to the porosity of the structure and to the lack of any substantial number of fibers running perpendicular to the orientation of the fiber layers. Without fibers diving deep into the structure from the surface the construct does not have the guidance cues necessary to direct cells to enter the fibrous mats.

To overcome the limitations seen in terms of cell seeding and cell infiltration to allow 3-D distribution of cells in electrospun scaffolds, there have been several techniques developed in hopes of improving or accelerating cellular infiltration/distribution. The first of is these was developed by Stankus et al. in which they electrosprayed media droplets containing cells in to the scaffold as the electrospun fibers were being deposited on the mandrel [9]. This technique was successful at creating electrospun scaffolding with high cell viability and seeding density throughout. However, a major limitation of this approach is exposing the cells contained within the developing scaffold to toxic organic solvents used in the electrospinning process which continue to evaporate from the fibers after deposition. More subtlety, when moisture is added to a scaffold in this manner it can greatly alter the deposition of fibers. This consideration is critically important when fiber arrays need to be deposited in very specific orientations, for example in highly aligned arrays. Additional concerns include the ability to maintain sterility in this multi-phase process and the length of time required for fabricating a scaffold in this manner. The time is critical to overall cell viability due to the presence of small amounts of cell culture media which will evaporate rapidly and lead to sample dehydration.

Another technique that attempted to enhance cell seeding and infiltration of electrospun scaffolds has been the use of hybrid scaffolds composed of both synthetic and natural polymers [10-13]. The synthetic polymers provide structural strength but possess no specific cell receptor binding sites such as integrin binding sites to provide the cells with binding sites for cell adhesion and migration. Thus, it is hypothesized that the addition of natural ECM polymers will provide the necessary integrin binding sites required to promote cell adhesion and infiltration. Unfortunately, the electrospun ECM protein scaffolds do not have sufficient structural integrity to be utilized in a majority of tissue engineering applications, thus, the structural integrity of the hybrid structure can be compromised by inclusion of ECM proteins. Further, while providing enhanced cell adhesion, the hybrid structures have had limited success in improving cellular infiltration. Another variation on a hybrid structure to enhance porosity is that of scaffolds composed simply of two distinctly different fiber diameters (μm and nm) in sequential layers [14]. The results of this study demonstrated that the thinner nanofibrous layers increased the cellular infiltration distance (generally limited to 200 μm unless perfusion cell seeding is done) into the scaffolds. This is simply due to large fiber diameters having lower packing efficiencies which in turn have higher scaffold porosities to allow cells to settle into the structure. However, a major concern of this scaffold fabrication technique is delamination, i.e. separation of the layers of the scaffold upon use.

The use of porogens in electrospun scaffolds has also been attempted. The first of these variations was demonstrated by Zhang et al. in which they electrospun a blended solution of polycaprolactone (PCL) and gelatin without cross-linking the scaffolding which meant a large percentage of the gelatin was dissolved when immersed in an aqueous media [15]. The dissolution of the gelatin made the scaffolds more readily infiltrated by cells as compared to PCL alone. This was followed by Baker et al. in which poly(ethylene oxide) (PEO) was electrospun and intermingled with simultaneously electrospun PCL fibers with the water-soluble PEO “sacrificial fibers” removed by post-processing submersion in water [16]. The results showed that greater the PEO in the scaffold, the greater the cellular infiltration (majority of cells remaining in the upper 25% of the scaffold) but at the expense of a significant reduction in the scaffolds' modulus and maximum stress. Nam et al. designed a system that introduced salt crystals to the Taylor cone region that allowed co-deposition of the crystals amongst the fibers that were removed by post-processing submersion in water to create void areas that resulted in enhanced cellular infiltration [17]. The major concerns with this technique are the uneven distribution of the crystals and loss of scaffold integrity (macroscopic scaffold layer delamination).

In sum, over the last decade, the use of electrospun tissue engineering scaffolds has met with mixed results primarily due to a lack of availability of scaffolds which promote cell infiltration and yet retain sufficient structural integrity for us in the formation of 3-D tissue. Attempts at increasing scaffold porosity have generally compromised scaffold mechanical integrity and have not demonstrated any substantial improvements with respect to the extent to which cells infiltrate the constructs.

### SUMMARY OF THE INVENTION

The present invention provides improved scaffolding for tissue engineering and/or for use in regenerative medicine, as well as for other applications. The scaffolds are formed by electrospinning, but, in contrast to prior art electrospun scaffolds, they possess two seemingly contradictory properties by having both 1) porous regions which permit ready infiltration by cells, and 2) dense regions which are less amenable to cell infiltration but which provide ample structural integrity to the scaffolds. Scaffolds with these properties are made using a mandrel that is perforated. During the electrospinning process, as fibers are deposited onto the perforated mandrel. Air emanating from the perforations is introduced into the developing layers of fibers that are located at or near the perforation, causing the mat of fibers in those areas to be less dense, creating regions of increased porosity. In contrast, fibers deposited on solid, non-perforated sections of the mandrel (e.g. located between and adjacent to the perforations) are, in comparison, densely packed. The resulting scaffold thus contains regions in which the fibers are porous and regions in which the fibers are densely packed, all within a single contiguous, seamless structure prepared during a single deposition event, i.e. advantageously, multiple deposition steps and/or processing steps are not required.

It is an object of this invention to provide an electrospun material comprising regions of densely packed electrospun fibers which are not permeable to cells and porous regions which are permeable to cells.

It is another object of this invention to provide an electrospun material comprising regions of densely packed electrospun fibers which are not permeable to cells and porous regions which are permeable to cells, wherein said electrospun material is formed by depositing incipient electrospun fibers onto a perforated mandrel while expelling a gas out of perforations in the perforated mandrel.

It is a further object of this invention to provide an artificial tissue or organ, comprising 1) electrospun scaffolding material comprising regions of densely packed electrospun fibers which are not permeable to cells and porous regions which are permeable to cells; and 2) a plurality of cells of interest associated with said electrospun scaffolding material. In one embodiment, at least a portion of the plurality of cells of interest are capable of carrying out at least one function of a tissue or organ of interest. In another embodiment, the plurality of cells of interest are comprised of a single type of cell. In yet another embodiment, the plurality of cells of interest are comprised of more than one type of cell.

The invention further provides an artificial tissue or organ formed by exposing electrospun material comprising regions of densely packed electrospun fibers which are not permeable to cells and porous regions which are permeable to cells to a plurality of cells of interest. The step of exposing is carried out in a manner that permits at least a portion of the plurality of cells of interest to infiltrate said electrospun material at the porous regions which are permeable to cells. In one embodiment, the step of exposing is carried out in vitro. In another embodiment, the step of exposing is carried out in vivo.

The invention further provides a mandrel for electrospinning fibers. The mandrel comprises a perforated support for receiving incipient electrospun fibers. In one embodiment, the perforations are arranged in a uniformly distributed pattern over the surface of said support. In another embodiment, the perforations are arranged in a non-uniformly distributed pattern over the surface of said support.

The invention also provides a method for forming electrospun material comprising regions of densely packed electrospun fibers which are not permeable to cells and porous regions which are permeable to cells; the method comprises the step of depositing incipient electrospun fibers on an outer surface of a perforated mandrel while directing a gaseous medium under pressure through perforations in the perforated mandrel toward the outer surface. In some embodiments, the gaseous medium is air.

The invention also provides an electrospinning system which comprises: 1) a source for generating incipient electrospun fibers during an electrospinning process; 2) a perforated mandrel for receiving the incipient electrospun fibers during an electrospinning process; and 3) a gaseous medium pressure source for directing a gaseous medium under pressure through perforations in the perforated mandrel during an electrospinning process.

The invention further provides a method of in situ tissue regeneration, comprising implanting into a subject in need thereof a scaffold comprising regions of densely packed electrospun fibers which are not permeable or have low permeability to cells and more porous regions which are permeable to cells. In one embodiment, the scaffold is formed by depositing incipient electrospun fibers onto a perforated mandrel while expelling a gas out of perforations in the perforated mandrel.

### BRIEF DESCRIPTION OF THE DRAWINGS

FIG. 1. Schematic of an exemplary electrospinning system of the invention with a perforated mandrel.

FIGS. 2A and B. Schematic views of a perforated mandrel with accumulating electrospun fibers. As can be seen, in the areas of the mandrel where perforations are present, fiber deposition is less than in areas where the perforations are absent. A, local deposition of fibers in and around perforations; B, regional view of an embodiment in which perforations are clustered.

FIG. 3A-C. A, Schematic representation of a regional, cross-sectional view of an exemplary cylindrical scaffold with areas of dense fiber packing and areas of sparse fiber packing; B, view of a micro-section of A with arrows indicating spaces through which cells can infiltrate the material, or spaces in which an agent of interest can be deposited; C, schematic representation of a side view of an exemplary cylindrical scaffold showing areas of dense fiber packing and areas of relatively high porosity where fibers are less densely packed. The arrows show possible gaps between fibers where cells may infiltrate the scaffold.

FIG. 4A-E. Schematic views of outer surfaces of exemplary perforated mandrels. Diameters of perforations are A, 250 microns (1 mm apart at 60° offset between rows); B, 500 microns (1 mm apart at 60° offset between rows); C, 750 microns at relatively low density (1.5 mm apart at 60° offset between rows); D, 750 microns at higher density (1 mm apart at 60° offset between rows); E, 1000 microns at relatively high density (2.17 mm apart at 30° offset between rows). We note that mandrel perforations and mandrel shape may be varied to achieve a variety of construct architectural features. For example, rectangular and square mandrels with slots, or perforations in square or oval configurations, are also possible

FIG. 5. PCL graft explanted with graft wall basically devoid of cells.

FIGS. 6A and B. Mandrel System (B) and a close-up (A) of the perforated mandrel (0.75 mm pores).

FIGS. 7A-C. Inside (A) and outside (B) micrograph of scaffold of electrospun PCL from 100 kPa applied to the perforated mandrel (Magnification=28×). (C) Representative micrograph for the solid mandrel internal and external surfaces (Magnification=100×).

FIGS. 8A and B. Representative outside surface of no applied pressure to the mandrel or the solid mandrel (A) and at 100 kPa (B) applied pressure for the electrospun PCL over a perforation (magnification=3,000×).

FIG. 9. A seeded (fiber orientation top to bottom) scaffold demonstrating cellular infiltration. Top was the seeded surface (scale bar=100 μm).

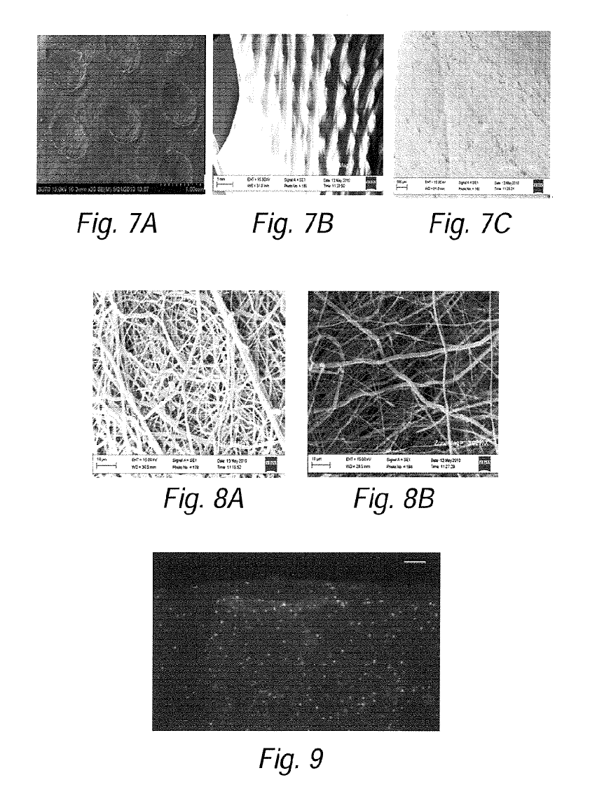

### DETAILED DESCRIPTION

Improved electrospun materials, which may be used as scaffolds for tissue engineering and/or for use in regenerative medicine, and methods and systems of making the same are described herein. In contrast to electrospun materials described in the prior art, the materials of the invention have both 1) porous regions which permit infiltration by cells, and 2) dense regions which are less amenable to cell infiltration but which provide necessary structural integrity to the scaffolds. This type of micropatterning provides or produces is regions of varying fiber density and can also be used to vary the relative thickness of fiber layers in specific domains of a tissue engineering scaffold. And materials with these properties are formed by a single electrospinning step which does not require additional processing e.g. to remove sacrificial fibers, salt crystals, etc. When used e.g. as scaffolding for tissue engineering applications, the materials advantageously allow cells to infiltrate the scaffold by migrating (or actively seeded under pressure in an electric field or by simply “falling”) into the pores located in porous regions of the scaffolding. Without being bound by theory, it is believed that once cells have entered the porous areas produced by the air impedance system, they migrate quickly along the fibers, forming an extended 3-dimensional network of cells. The less dense areas allow cells to enter the scaffold, the cells can then migrate efficiently along the fiber networks laterally and parallel to the surface of the construct by moving along the fibers and thereby populate the scaffold. In some embodiments, such networks (or groups or masses) of cells approximate the appearance and/or functional capabilities of a tissue or organ of interest and are thus useful for tissue engineering applications. The original shape of the scaffolding is largely retained throughout this process of cell entry into the pores and subsequent migration due to the preservation, in the material, of regions which are not porous or which are less porous. While these regions may not allow or facilitate the entry of cells into the material, they are important because they help to preserve the strength and mechanical integrity of the scaffold. Further, the density is such that the migration of cells along the length of the fibers is still possible, once they have infiltrated the scaffold at the porous regions. Due to their robustness, the materials of the invention can be readily manipulated as needed without damage, and can be used in applications where pressure is exerted on the structure, e.g. they may be used to replace blood vessels or various other organs or tissues for which it is necessary or advantageous to retain a particular shape, either in vitro or in vivo.

Scaffolds with these two seemingly contradictory properties (porosity and mechanical integrity) are made by introducing air into layers of incipient (i.e. newly formed or nascent) fibers as they are deposited on a support after drying in flight. This is accomplished using a mandrel that is selectively perforated with a defined pattern of perforations. As the fibers are deposited on the mandrel, air flows out of the perforations, and, in effect, the air flow interferes with fiber deposition so that the fibers in the vicinity of the perforations overcoming the ground effects of the mandrel and are physically “pushed” aside as they begin to approach the mandrel. As a result, the fibers cannot pack as densely as they would in the absence of air flow. Instead, spaces or pores are formed between or among the fibers. However, this occurs only at or near the perforations. On the intervening solid portions or sections of the mandrel, fiber deposition occurs as in traditional electrospinning, i.e. the fibers deposit in a densely packed, electrospun mat. The resulting scaffold thus contains regions of porosity and regions in which the fibers are densely packed, all within a single contiguous, seamless structure. The sizes and numbers of the perforations, the rate of air flow, and the spatial arrangement of the perforations can be adjusted as described below to produce an electropun material with desired properties. This invention concentrates on describing the manner in which an air impedance system can be used to reduce porosity by actively and selectively reducing fiber deposition in the vicinity of the output pores present in the ventilated mandrel. One skilled in the art of electrospinning will recognize that under certain conditions that it may be advantageous to not use any air flow at all to produce a scaffold on this type of mandrel. Edge effects produced by the ventilations present in a conductive mandrel will cause fibers to deposit in patterns that are different than the surrounding areas that comprise the solid aspects of the mandrel. This is because electric charges concentrate on such edges; this effect can be exploited to cause fibers to deposit in different orientations and even in an aligned fashion in a regional pattern within an electrospun scaffold. This type of deposition can be used to impart unique material and functional properties onto the structure. Thus, the mandrel design itself represents an important consideration in this invention as a means to control fiber orientation in a regional manner on a ventilated mandrel due to these edge effects.

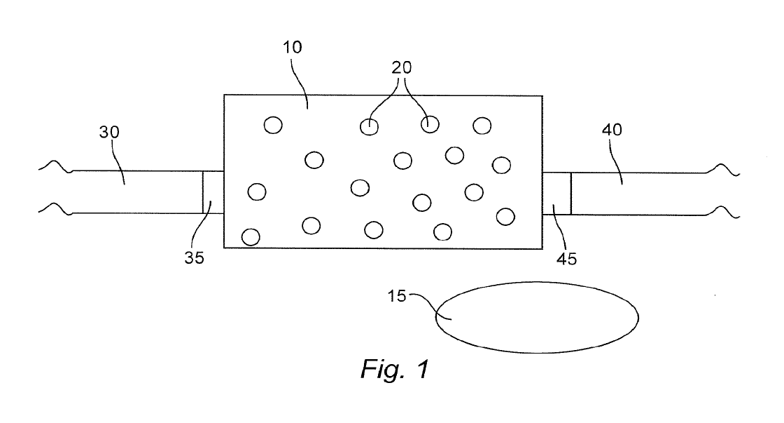

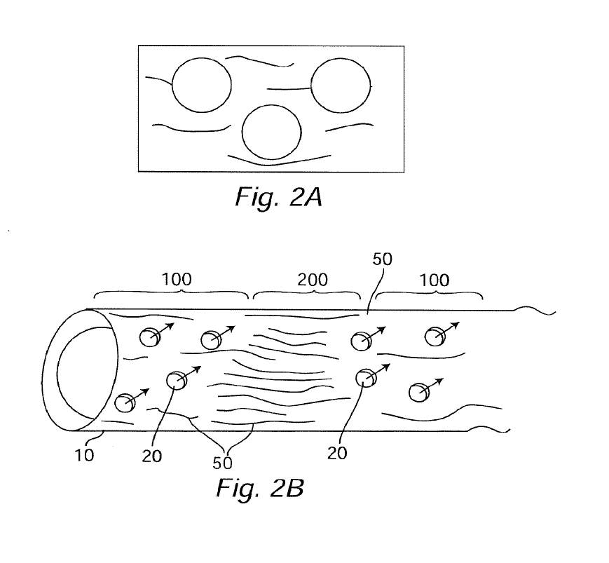

FIG. 1 is a schematic which shows exemplary mandrel 10 (which in this example is cylindrical) with pores 20 through which air (or another gaseous medium or carrier) can exit. The actual deposition process is schematically illustrated in FIG. 2, which shows mandrel 10 (which in this example is also cylindrical) with pores 20 through which a current of air is being driven while electrospun fibers 50 are being and/or have been deposited. The direction of flow of the gaseous carrier (e.g. air) out through perforations 20 in mandrel 10 is shown by the arrows. As can be seen, a region of relatively dense fiber deposition 200 is located in an area that does not contain perforations, whereas areas with perforations contain regions of relatively less dense fiber deposition 100. In this exemplary depiction, the perforations on the mandrel are not evenly distributed but occur in patches. Those of skill in the art will is recognize that the perforations on the mandrel, and hence the porous regions of the material, may be present in, for example, “patches” along the mandrel (as depicted in FIG. 2), or may be arranged longitudinally along the length of the mandrel, or may be uniformly distributed over the surface of the mandrel, or may be in some other desired pattern or configuration. The spacing between pores 20 and the size of the pores 20 may me uniform or variable depending on the structural material being fabricated.

As disclosed herein, the air impedance system is effective at selectively decreasing fiber density in the vicinity of the pores in the mandrel. However, in some embodiments, this system can be” inverted” and a negative air pressure can be applied across the pore spaces. Under these conditions, fibers are pulled inwards towards the holes (and possibly even into the perforations), in effect, creating a structure with raised areas or “bumps” that correspond to the pore sites. The micropatterned bumps may extend into the mandrel and when the structure is removed from the mandrel, they project above the adjacent areas of the scaffold. This type of structure can also be achieved by injecting air into the impedance system such that fibers are largely restricted from depositing near the pores and allowing the adjacent areas to build up piles or mountains of fibers. One distinct advantage to this type of structure is that when the air flow is reduced or reversed during the electrospinning procedure, fibers will continue to deposit onto the “slopes” of the fiber mounds, thereby providing additional guidance cues to direct cells to migrate along and enter the deeper domains of the construct. Fibers deposited in this manner would run e.g. from the surface of the mounds and down the slopes towards, and even into, the pore sites. Such micropatterns can play a role in directing cellular distribution and function. For example, this type of scaffold would resemble the rete pegs that make up the junction of the dermis and epidermis in the skin.

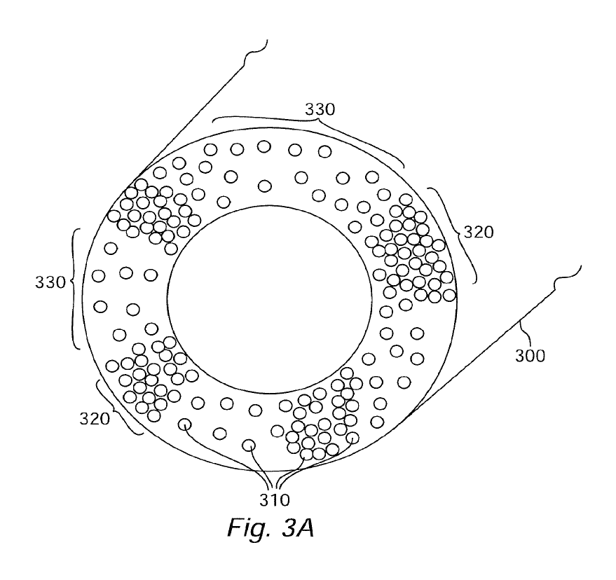

FIG. 3A depicts a schematic cross-sectional view of cylindrical (tubular) scaffold 300 formed using air impedance electrospinning in which air is injected into the mandrel during the spinning process. In this figure, the “cut” cross-sectional ends of individual fibers 310 are shown as present in regions of dense fiber packing 320, or in less densely packed porous regions 330. FIG. 3B shows a micro-view of the cross section of an edge of a small porous section of material. The arrows show spaces between fibers 310 where e.g. cells may infiltrate the material. FIG. 3C shows a schematic representation of the outer surface of a scaffold 300 with regions of dense fiber packing 320 and porous regions 330. Arrows indicate interstices between loosely packed fibers through which cells (or other materials or substances) can infiltrate the structure.

Those of skill in the art will recognize that the precise pattern of porosity vs density can be altered by designing a desired pattern of perforations on the mandrel that is used to prepare the electrospun material. For example, variations may be made in the shape and size of the mandrel and/or in the size of the perforations, their placement on the mandrel, the pattern of perforations (e.g. evenly distributed over the entire surface, or in lines, or only in distinct circumscribed sections of the mandrel, etc., according to the desired use of the material. This system can be adapted and used with a mandrel that is not designed to rotate so that larger flat sheets can be prepared.

Similarly, the rate of flow of the gaseous carrier, usually air (but in some applications could be nitrogen, carbon dioxide, hydrofluorocarbons, alkanes, or other gases), through the perforations can be adjusted to achieve a desired level of porosity, and can be adjusted in concert with the size and/or shape and/or density and/or pattern of the perforations. When utilizing this technology, those of skill in the art will appreciate that, if no air is expelled through the perforations, then the electrospun fibers will be deposited and pack together in a manner that resembles a conventional electrospun mass of densely packed fibers. On the other hand, if sufficient air is blown through the perforations and in and around the fibers as they deposit, an extremely loosely packed structure with an overall “cotton ball” type porosity can be made. In order to achieve desired densities in between these two extremes, a practitioner of the invention will adjust the flow rate, depending on the material(s) that is/are being electrospun, and the design requirements (e.g. porosity, strength, etc) for a desired application. For example, generally, a flow rate of from about 1×10⁻⁵ liters/second per pore or less to about 1×10⁻² liters/second per pore or more, or from about 5×10⁻⁴ to about 7.5×10⁻³ liters/second per pore, or from about 1×10⁻³ to about 5×10⁻³ liters/second per pore. Any level of air flow may be employed so long as the objective of the procedure is achieved, e.g. so long as the desired level of interference with (impedance of) fiber deposition occurs. However the flow rate will vary depending upon the specific spinning conditions as there is in a dynamic interaction between fiber size and the strength of the electric field and the rate of air flow through the mandrel pores. Also, the density of the gas or fluid injected into the impedance system will play a role on how fast and under what pressures it must be inject to achieve the desired effects. Further, the flow rate may also be varied according to mandrel shape and size, etc. Those of skill in the art are well acquainted with sources of gaseous carriers (e.g. air, nitrogen, oxygen, argon, etc.), especially pressurized sources, from which gas egress rate can be controlled, and also with other mechanisms for controlling the rate of flow. Any suitable means for controlling the flow may be employed in the practice of the invention.

In addition, gaseous media at different temperatures and/or densities may be utilized to influence fiber deposition. In some embodiments, the medium being ejected from the pores may be partially supplemented (e.g. at least 0.1%) or even completely (e.g. 100%) by a solvent system for the fibers. When processed in this way, the flow of gas containing a fiber solvent would partially or completely dissolve or degrade the fibers as they are deposited over the pore sites. This technique would be used to selectively solvent weld the fibers near the pores together. An example of this is, when PCL spun from TFE is used to form the fibers, the air impedance system can be supplemented with TFE and/or chloroform. Once applied through the air impedance system, this will partially degrade fibers in the near vicinity of the pores present in the ventilated mandrel. Conversely, agents that impact the chemical structure in other ways may be used. For example, fibers might be engineered to contain reagents that react with materials ejected from the pores. All the fibers might have such a reagent in them but, the fibers near the impedance sites would, due to their proximity to the pores, be preferentially exposed to a chemical in the medium, thereby causing the desired reaction to take place selectively in regions located near the pores. This technique might be used to impart regional functional differences in the scaffold. One skilled in the art will recognize that the converse situation is also possible, e.g. chemical reactions can be designed to remove or add various functional or physical properties to the fibers near the pores by adding suitable reactants to the material(s) from which the fibers are formed and to the medium that is ejected through the perforations of the mandrel.

The invention also provides perforated mandrels. Such mandrels comprise a support (generally a rigid support) for receipt of nascent electrospun fibers, i.e. at least one surface on which newly formed electrospun fibers (which have substantially dried during flight) are deposited. The support may be made from any suitable material (e.g. made from various metals, alloys, or synthetic materials such as plastics, etc.). In some embodiments, the support is made from stainless steel. The support which receives the fibers is perforated, i.e. the support comprises an inner and outer surface with holes or pores extending through the mandrel. Typically, the inner surface of the mandrel defines (surrounds, circumscribes, etc.) a cavity or lumen, i.e. at least a portion of the mandrel is hollow, usually a portion at which perforations are located. The perforations may occur uniformly over the entire mandrel, however, this is not always the case. In some embodiments, only one side of the mandrel is perforated, or only a selected section or sections is/are perforated. Various patterns of perforation may be present in order to produce an electrospun material with a desired corresponding pattern of porous regions. Likewise, the shape and/or other characteristics of the mandrel itself can be varied as discussed below so as to produce material with a desired shape and/or characteristics. When air is to flow through the perforations, generally it is introduced into the lumen of the mandrel and flows out through the perforations toward the outer surface of the support, and it is the outer surface of the mandrel that receives the nascent, newly (initially) formed electrospun fibers, with the air disrupting fiber deposition as described above to create pores or spaces between or among the fibers, but not (or at least less so) in areas of the mandrel that do not have perforations. However, in some embodiments, air may be introduced through the perforations via tubes, e.g. tubes which fit into the perforations on the side of the support opposite to that on which the fibers deposit, or tubes which extend through the perforations in the support. This embodiment allows the introduction of the gaseous medium to different groups or clusters of perforations at different rates or pressures. For example, a gaseous medium may be introduced into the perforations in one section of the mandrel at a rate that is higher than at another section of the mandrel, thereby creating porous sections with differing porosities within a single piece of electrospun material. Alternatively, or in addition, different types of gaseous media, or gaseous medias with differing additives of interest, may be expelled to different perforations, or to different groups, patterns or clusters of perforations. For example, reagents or other agents of interest as described herein may be added to the medium flowing through tubes at some perforations but not to others, providing a customized distribution of active agents at the porous regions of the electrospun material.

The shape of the mandrel, and hence of the electrospun material, may be any that is desired. In some embodiments (e.g. when making scaffolds for vascular grafts), the mandrel is usually cylindrical and the electrospun material is also generally cylindrical or tubular. However, in other embodiments, the mandrel surfaces may be curved but tapered to form a cone, or ovoid, or cuboid (e.g. forming a rectangle in cross-section), or even a completely irregular yet forming a desired shape. The dimensions of the mandrel may vary with the design goals and type of material that is electrospun and/or its intended use, so that wide variations in volume, surface area, diameter, diagonal and/or axis lengths, etc. may vary. However, frequently the mandrel is cylindrical in shape with dimensions on the order of: a length from about 100 to about 1000 mm, or from about 300 to 500 mm and a diameter of from about 1 mm to about 1000 mm or more.

In some embodiments, particularly those associated with mass production, and/or with the production of relatively large sheets of electrospun material, the support that receives the nascent fibers may be a flat “conveyor belt” style support that may or may not move during deposition, i.e. a true moving conveyor belt may be used, or what is used may be simply a large support that is stationary, or that undergoes translational movement(s), or that oscillates from size to side, or that gyrates, etc., depending on the desired pattern of deposition. Such embodiments may be used especially when large electrospun mats are formed, e.g. with dimensions on the order of inches, feet, centimeters, meters, etc., or even larger. Large sheets of electrospun material may be formed and used “as is”, or the sheets may be trimmed or cut to a specified size for use, e.g. as filters, etc., or may be further shaped by folding, rolling, etc., as appropriate.

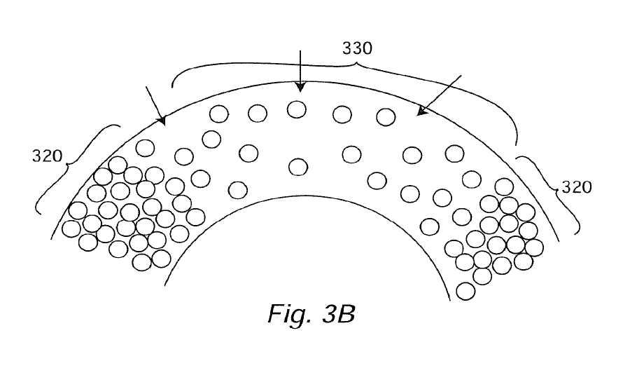

The perforations that are present in the mandrel may be of any suitable size and shape, and may be present at any desired frequency on the surfaces of the mandrel. Generally, the perforations are roughly or substantially cylindrical, with a diameter (e.g. usually an average diameter) ranging from about 100 to about 2000 microns, or from about 200 to about 1500 microns, or preferably from about 250 to about 1000 microns. Perforations with a square, rectangular, triangular or other angular configuration can be used to increase the edge effects observed in an electric field, and these patterns can be used to further modulate the pattern of fiber deposition. All perforations in the mandrel may have substantially the same diameter or average diameter, or they may vary, i.e. some sections may have perforations with larger or smaller diameters than those that are present in other sections of the mandrel. Views of an outer surface of exemplary perforated mandrels are shown in FIGS. 4 A-E depict schematic representations of arrangements of perforations on a mandrel surface. It should be recognized that the perforations may be polygonal, star shaped, slotted, rectangular, or any other shape which may yield desirable structural properties in the material which to be electrospun. Further, the channels need not be straight but may be tunneled through the support at an angle, and combinations of these different designs of perorations may be used in a single mandrel. The perforations may be formed by any of several known methods, e.g. by etching using techniques similar to those use for the manufacture of semiconductors, or be drilling, or by pouring molten material into a suitable support, etc.

The dimensions of the electrospun materials that are formed using the methods and apparatuses described herein may vary widely, depending on the design requirements, their intended use, and how they are made. Generally, the materials (e.g. scaffolds) that are formed on the mandrel have dimensions similar to those of the mandrel on which they are formed. In an embodiment, e.g. for use as a vascular graft, the length is on the order of from about 1 cm or even less to about a meter or longer, as required. The shape of a vascular graft may be any that is useful, e.g. cylindrical, cone-shaped, etc. The thickness of the electrospun material will vary depending on the amount or number of layers of fibers that are deposited, the dimensions of the fibers, amount of porosity that is introduced, etc., and may be varied to accord with desired characteristics of the material being formed. Further, modifications may be made to the electrospun material after formation, e.g., as noted above, a tubular scaffold may be cut to form a sheet, or cut to form multiple smaller scaffolds, or multiple scaffolds may be joined together, or a scaffold may be trimmed to a desired size or shape, etc. In addition, the generally tubular material formed on the mandrel can be cut to form flat sheets of electrospun material with dense and porous regions.

The impedance system also offers the opportunity to fabricate unique blended materials and gradients of materials. For example, in one embodiments, this may be achieved by using a mandrel with an inner sliding core that is hollow and not ventilated except at either end (although other configurations are also encompassed). One end of the internal core may be connected to an air supply and the other is left open. At the onset of spinning, the inner core may be placed at one end of the outer ventilated mandrel. By injecting a large volume of air, fibers can be nearly completely excluded from depositing in the domains near where air is being injected by the inner cylinder, i.e. where air flow is extremely high. By moving the inner cylinder with respect to the outer ventilated cylinder and/or by attenuating air flow, fibers can be allowed to deposit in different places. This approach may be used to reduce fiber deposition at the distal end(s) of the ventilated mandrel as a method to produce a gradient of mechanical properties and/or to tailor the compliance of the electrospun material in specific domains. One fiber type may also be spun (e.g. a fiber of a specific size, composition, etc.) initially and then attenuated as the inner mandrel is moved and a new fiber type is spun. This technique can be used to produce a gradient of fibers with respect to size, identity etc., e.g. from one end of the outer ventilated cylinder to the other, or in specific domains. We note that gradients or selective fiber deposition on a target can be produced by masking the target mandrel. This can be achieved by placing a mask (physical barrier) between the source electrospinning solutions and the target mandrel thereby physically blocking the deposition of fibers onto a portion of the target mandrel. That mask can then be moved to allow fibers to deposit onto different aspects of the mandrel. However, the air impedance technique affords more subtle control over the fiber deposition process. Masking a target mandrel can not obviously be used to regulate porosity in the highly selective manner that can be achieved with an air impedance system.

We also note that, by using an impedance system, very high flow rates can be used to nearly completely attenuate fiber deposition over the ventilated areas of the mandrel. By spinning under these conditions and then stopping the air impedance system and spinning more of the same polymer (or any number of different polymers and/or blends of polymers) unique structures can be produced. For example, fibers of PCL might be spun and excluded from the pore sites in the ventilated mandrel. Then, by stopping the air flow, the entire surface might be overcoated with fibers of collagen or other material. The resulting construct has a backbone of PCL fibers coated with collagen, and the ventilated sites will be nearly exclusively collagen.

An air impedance mandrel may also be used to manipulate the structural and/or functional properties of the scaffold at the conclusion of the spinning process. For example, fibers might be spun over the surface of the target ventilated mandrel with our without air flow. Next the mandrel might be injected with some material that is designed to exit the mandrel ventilation holes and preferentially enter the scaffold at those sites. This may be done to produce a scaffold that contains an electrospun backbone with different materials impeded in it at areas corresponding to the ventilation sites. For example, a bone implant might be designed to have a PCL collagen co-polymer fiber backbone. Bone cement can then be injected into the mandrel and allowed to enter the scaffold though the ventilation pores. This particular construct would then contain fibers all over, but the fibers near the ventilation pores would be enveloped in the bone cement. This type of arrangement might also be used to treat domains near the pores with other materials, for example, cross linking agents, either in liquid or a gas phase (for example glutaraldehyde in vapor phase). This approach allows for regional differences in cross linking. One skilled in the art will recognize that it is also possible to suck or draw substances of interest in through the ventilated mandrel to supplement or otherwise manipulate its composition, functional and/or structural properties.

The electrospun materials that are formed on the mandrel comprise sections or portions which are porous and other sections or portions which are relatively non-porous. Generally, a “porous” section of the material has a pore size in the range of from about 5 to about 150 microns (or greater, depending on the desired use of the material), and preferably from about 5 to about 60 microns, especially for biological applications designed to allow the entry of cells and other materials in a size range of from about 5 to about 50 microns, and usually about 5 to about 30 microns, to infiltrate the structure. In contrast, a non-porous or “dense” section generally has a pore size of less than about 5 microns.

The porous regions of the scaffold allow cells or other materials or substances of interest to enter into the scaffold at those regions. Those of skill in the art will recognize that such cell entry may be brought about by various means, e.g. by placing the material in an environment (in vitro or in vivo) where motile or growing or dividing cells will encounter the porous regions and tend to migrate, or “fall” or grow into the pores. Alternatively, cells may be mechanically introduced into the material, e.g. by rinsing or otherwise coating the material with a solution of cells. The cells may be actively injected through the same or different ports of the air impedance system into the inner surface of the mandrel. By suspending them in a suitable medium and passing them into the inside of the ventilated mandrel that has had a scaffold spun onto it, the cells can be applied to the porous areas from the inside of the mandrel. If this seeding method is done under pressure, cells can be induced to flow into the porous regions of the scaffold. Further, the materials that are incorporated into the material need not be cells. For example, various chemicals; coloring agents; medicaments; drugs; nutrients; various polymers; biological molecules (e.g. proteins, nucleic acids such as DNA, RNA, lipids, attractants such as cell attractants, etc.); metal particles (e.g. catalysts); activated charcoal (e.g. for filtration), bead or nanoparticle structures (e.g. unloaded for use in capturing or scavenging substances of interest, or loaded with one or more agents of interest, e.g. cells and/or drugs, growth factors, cytokines, etc.; dendrimers (either attached to the fibers or put into porous sections); functionalized dendrimers; hyperbranched polymers; electrospun materials may be permeated with gels with or without active agents such as drugs, bioactive materials such as growth factors, cDNAs, DNA, sRNAs, viruses, bacteria, chemokines, sugars, attractants, e.g. attractants for cells, agents that restrict cell infiltration (so that porous areas remain porous but relatively devoid of cells); biological molecules as described above, various small molecules; cross linking agents, powders designed to undergo hardening such as bone cement; therapeutic reagents including pharmaceuticals; etc. Such substances may be incorporated into the electrospun material of the invention, e.g. by soaking or rinsing the material in a solution of the substances, or even by loading the substances into the stream of air that causes scaffold porosity so that they are deposited during fiber deposition. Due to the unique structure of the material, and depending on their size, such substances may diffuse or otherwise preferentially enter the porous regions of the material. Nevertheless, the material retains its overall strength, shape, integrity, etc. due to the presence of the relatively non-porous regions.

The electrospun materials of the invention retain their structural integrity and strength in spite of the presence of porous regions therein. Those of skill in the art will recognize that the precise attributes of an electrospun material of the invention, including but not limited to size, shape dimensions, strength, etc., may be varied in order to meet design requirements in terms of properties for a desired application.

Fiber orientation on the target mandrel is generally regulated by spinning conditions. For example, when a slowly rotating mandrel is used, fibers will collect in a random fashion over the surface of the target mandrel. This will occur with or without air flow through the ventilated target mandrel. By increasing the rate of mandrel rotation (increased rotational velocity), fibers can be induced to deposit in an aligned manner and in a circumferential pattern about the target mandrel. If a non-conductive ventilated target mandrel is suspended between two grounded poles fibers as in a two pole air gap electrospinning system, the fibers can be induced to collect along the surface of the mandrel in parallel with the long axis of the cylindrical mandrel. [See: Jha B S, Colello R J, Bowman J R, Sell S A, Lee K D, Bigbee J W, Bowlin G L, Chow W N, Mathern B E, and D G Simpson. Two pole air gap electrospinning: Fabrication of highly aligned, 3D scaffolds for nerve reconstruction. Acta Biomaterials 7:203-215 (2010)]. Fibers can also be induced to collect on the target mandrel if the mandrel is placed between a source of polymer and a separate ground. Under these circumstances, fibers may be induced to form as a polymer leaves the source reservoir and passes towards the ground, and fibers will collect on the ventilated mandrel if it is placed in a position between the source of polymer and the ground.

Exemplary materials, usually polymers, which may be used to manufacture the selectively or partially porous electrospun materials of the invention include but are not limited to: polyurethane, polyester, polyolefin, polymethylmethacrylate, polyvinyl aromatic, polyvinyl ester, polyamide, polyimide, polyether, polycarbonate, polyacrilonitrile, polyvinyl pyrrolidone, polyethylene oxide, poly (L-lactic acid), poly (lactide-CD-glycoside), polycaprolactone (PCL), polyphosphate ester, poly (glycolic acid), poly (DL-lactic acid), and some copolymers (e.g. PLA co-polymers of PGA PLA, polyesters, and native proteins such as collagens, gelatin, fibronectin, fibrinogens, recombinant proteins and other natural and synthetic proteins and peptide sequences); biolmolecules such as DNA, silk (e.g. formed from a solution of silk fiber and hexafluoroisopropanol), chitosan and cellulose (e.g. in a mix with synthetic polymers); various polymer nanoclay nanocomposites; halogenated polymer solution containing a metal compounds (e.g. graphite); memory polymers including block copolymers of poly(L-lactide) and polycaprolactone and polyurethanes, and/or other biostable polyurethane copolymers, and polyurethane ureas; linear poly(ethylenimine), grafted cellulosics, poly(ethyleneoxide), and poly vinylpyrrolidone; solutions of polystyrene (PS) in a mixture of N,N-dimethyl formamide (DMF) and tetrahydrofuran (THF) poly(vinyl pyrrolidone) (PVP) composites; poly(L-lactide), poly(D,L-lactide), polyglycolide, polycaprolactone, polydioxanone, poly(trimethylene carbonate), poly(4-hydroxybutyrate), poly(ester amides) (PEA), polyurethanes, and copolymers thereof; various polyesters and acrylics; various colloidal dispersions; solutions with dispersed hydroxyapatite (HA) particles; polysulfone and a vinyl lactam polymers; dextrans; various charged nylons (e.g. nylon 66 for protein adhesion and other variants designed to adhere to RNA and DNA); nitrocellouse; dendritic poly(ethylene glycol-lactide); etc. These materials and electrospinning techniques and variants thereof (e.g. various applications of electrospun materials, various coatings, etc.) are described, for example, in issued U.S. Pat. Nos. 6,110,590; 7,887,772; 7,824,601; 7,794,219; 7,759,082; 7,615,373; 7,575,707; 7,374,774; 7,083,854; 6,787,357; 6,753,4541; and 6,592,623; and published US patent applications 20110150973; 20110148004; 20110143429; 20110140295; 20110135901; 20110130063; 20110123592; 20110092937; 20110091972; 20110079275; 20110072965; 20110064949; 20110052467; 20100310658; 20100291058; 20080159985; 20080038352; 20050192622; 20040116032; 20040009600; and 20030207638; the complete contents of each of which are hereby incorporated by reference, as are the references cited therein.

Many tissues are organized in a hierarchical pattern. For example, in the skin the epidermal layer is composed of cells. These cells sit on an underlying layer of connective tissue called the dermis. Ridges project from the dermis upwards into the overlying epidermis. These macroscopic structures, called rete pegs, are composed of microscopic scale fibers of connective tissue. By projecting upwards at intervals into the epidermis and transmitting small blood vessels into the vicinity of the epidermal compartment, the rete pegs reduce the diffusion distances needed to provide oxygen waste and nutrient exchange to the cells of the epidermal compartment. Rete pegs also increase the surface area of the epidermal dermal border, thereby strengthening the adhesion between these domains (reducing the chances that the epidermis will delaminate from the dermis during trauma). Unfortunately, rete pegs usually fail to reform when a burn is treated with a dermal template or skin equivalent. The border between the dermis and epidermis tends to be nearly linear. The micro-patterning that is possible with an air impedance based electrospinning as described herein makes it possible to deposit nano to micron scale fibers into hierarchical patterns that mimic biological structures such as rete pegs, providing a method to more closely recapitulate the native structure of skin in a dermal template or skin equivalent than has heretofore been possible. Micro scale structures that form higher orders of macro structure are also present in other tissue. For example, long bone is composed of a series of osteons. These structures resemble cylinders that are oriented in parallel with the long axis of the bone; each osteon has a central canal called a Haversian Canal that contains a blood vessel. Surrounding the central Haversian Canal are ostocytes imbedded in connective tissue matrix of compact bone, and these cells are arranged in a series of concentric circles. Many osteons are packed together to form the shaft of a bone. In bone engineering, the recapitulation of this structure using the electrospinning technology described herein can be used to more efficiently provide (e.g. in an implant used for bone regrowth, replacement, augmentation, etc.) the signals necessary to produce bone with a more normal profile of mechanical properties than does the use of unorganized implants such as those that are currently used. The production of matrices (supports, scaffolds, etc.) for tissue engineering of skin and bone are examples of how the present technology can be advantageously tailored to achieve a desired topology that is conducive to directing cell migration, attachment, and subsequent development into structures that resemble, or at least partially or fully fulfill the functions of, various tissues, organs etc. Those of skill in the art will recognize that this capability can be advantageously applied to the engineering of many other tissue and organ types which can also benefit from taking the microscopic and macroscopic topology of biological structures into account.

The electrospun materials described herein may be utilized for a variety of applications. For example, they may be used as stent coatings or vascular grafts, or as supports for the regrowth of new tissues or cells or even organs, or as nerve guides, or a bandages or dressings, skin mimetics, dermal and skin templates, dura mimetics and other connective tissues like ligament and tendon, in cosmetic surgery and/or reconstructive surgery, etc., either in vitro or in vivo.

In some embodiments, they are used for tissue engineering endeavors which use a combination of cells (which may be added exogenously to a support or may originate in a patient's body), engineering, materials, and (optionally) suitable biochemical and physio-chemical factors to improve or replace biological structures, particularly structures that are injured, damaged or missing, or that need to be removed and replaced. “Tissue engineering” covers a broad range of applications, but is generally associated with applications that repair or replace portions of or whole tissues (i.e., bone, cartilage, blood vessels, bladder, skin, etc.). Often, the tissues involved require certain mechanical and structural properties for proper preparation prior to and/or during in vivo use. “Tissue engineering” encompasses efforts to perform specific biochemical functions using cells within an artificially-created support system provided by e.g. a scaffold (e.g. an artificial pancreas, liver, kidney, etc.). The term “regenerative medicine” may be used synonymously with “tissue engineering”.

In some embodiments, a scaffold that is not pre-seeded with cells is implanted in a subject in need thereof to supply the structural properties of missing or damaged organs and/or tissues. For example, such scaffolds may be used as stents or stent coatings in blood vessels. In this embodiment of in situ regeneration, the cells which infiltrate the support come from internal body tissue, as do the physiological factors that interact with the cells (although drugs or active agents may also be added to the support before implantation, e.g. agents which stimulate angiogenesis). Cells from the recipient's body infiltrate porous areas of the scaffold after it is implanted and, using the scaffold as support, migrate within the scaffold and undergo cell division and differentiation within or on the scaffold, eventually forming a substitute tissue/organ (or a mass of cells that functions as a substitute organ or tissue) that has at least some beneficial attributes or capabilities of the organ/tissue that has been replaced, or whose function is being augmented.

In other embodiments, prior to implantation, the cells may or may not be derived from the recipient's body, at least not initially. Instead, the scaffold is “seeded” (“pre-seeded”) with cells capable of regenerating the function of the missing or damaged organ or tissue (or with cells which differentiate into such cells), e.g. a scaffold used as a vascular graft may be pre-seeded with cells that are or are capable of differentiating into cells that form blood vessels, and the pre-seeded scaffold is then implanted where it takes over or supplements the functions of the missing or damaged (e.g. diseased) organ or tissue. The support may be seeded with one type of cell or with a plurality of cell types. In other embodiments, the cell seeded scaffold may mature or develop into a structure that approximates or has at least some functional capabilities or attributes of the organ or tissue that it is to replace. In this embodiment, the original scaffolding may or may not be present in entirety at the time of implantation, i.e. the artificial organ/tissue may have formed on the scaffold and the entire structure, including the intact scaffolding, may be implanted; or the scaffolding may be partially or fully dissolved or disintegrated while still in vitro, leaving behind the artificial organ/tissue, which is then implanted. Alternatively, part or the entire original scaffold may be present upon implantation but may, with time, disintegrate once inside the body.

The materials and/or scaffolds of the invention may be used in applications which include but are not limited to: as stents and/or for bypass or other surgeries involving blood vessels and the circulatory system; to prepare “artificial” organs or clusters of cells which perform part or all of the function of an organ, e.g. heart, pancreas, liver, skin, skeletal muscle, cardiac muscle, intestine, bowel, esophagus, trachea and other hollow organs, nerve, bone, etc.

The materials of the invention may also have applications in other fields, e.g. manufacture of fabrics, electronics, etc. where is it useful to use a differentially porous or permeable electrospun material. For example, they may be used for various non-medicinal purposes including but not limited to uses for air or liquid (e.g. water) filtration, in energy systems, in batteries, in absorbent pads or padding, in sound barriers or insulation; etc.

The invention also provides an apparatus and/or system for fabricating the electrospun materials of the invention. Generally, such a system will include a perforated mandrel as described herein, together with a means of moving (usually rotating or spinning, but various translational movements are also contemplated) the mandrel, and a source of gaseous carrier, which is usually but not always air, as illustrated schematically in FIG. 1, where mandrel 10 with perforations 20 is shown as operably connected to rotation means 30 and air source 40. The mandrel itself may optionally comprise attachment mechanism 35 for attaching to rotation means 30, and an intake 45 for receiving e.g. air from air source 40. The system also comprises source of electrospun fibers 15.

The invention will be further understood in view of the foregoing Examples which should not, however, be interpreted as limiting the invention in any way.

### EXAMPLES

### Example 1

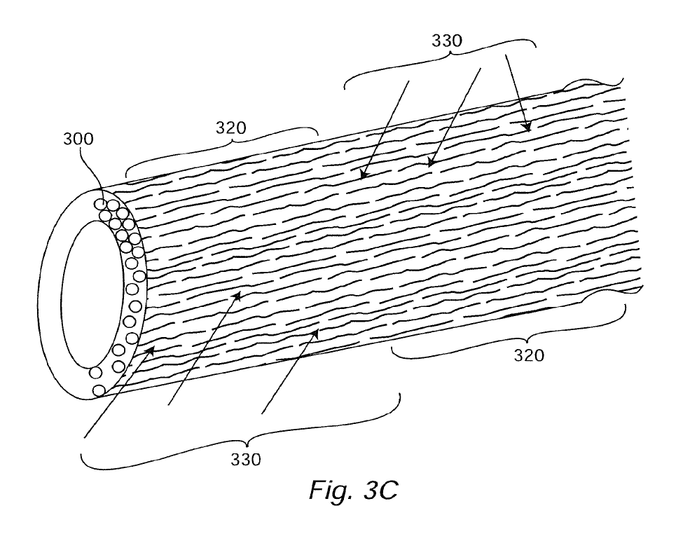

Previous studies have demonstrated the need for increased scaffold porosity and cellular infiltration ([9, 18, 19]). As one representative example, 1.5 mm inner diameter (I.D.) electrospun PCL (65,000 MW, Lakeshore Biomaterials) grafts were placed in a rat aortic inter-position model for up to 1 year. The grafts were composed of 480 μm diameter PCL fibers (graft wall thickness=500 μm). Histological evaluation at 12 weeks showed evidence of arterial regeneration (neo-intima and media) with minimal cellular infiltration into the graft wall structure (FIG. 5). Results showed tissue development only on the luminal and abluminal surfaces with no aneurysm formation. The lack of any evidence of aortic aneurysm in this model indicates that electrospun PCL has excellent potential in this type of application; however, in order to translate this type of construct into human use it will be necessary to greatly enhance cellular infiltration and 3D tissue development.

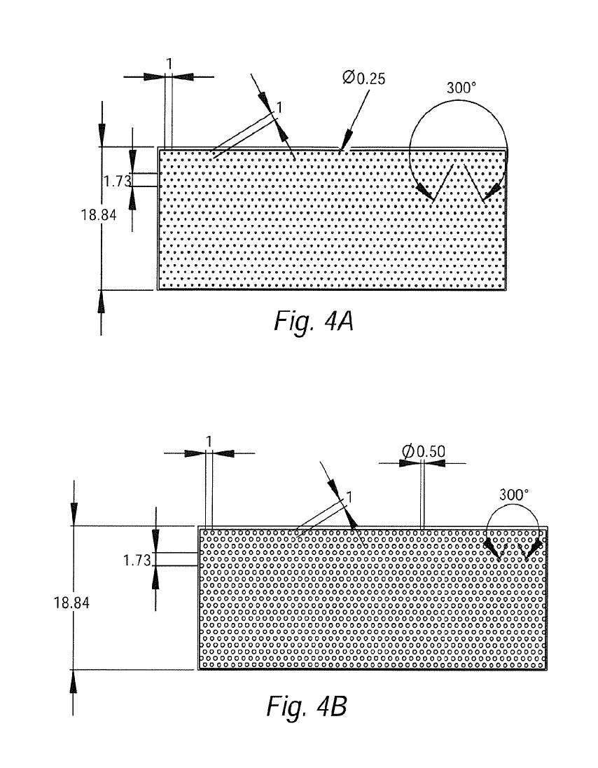

To overcome the cell infiltration limitations observed in scaffolds produced by conventional electrospinning, we have developed a novel electrospinning mandrel system to create a more open, porous structure by air-impedance electrospinning. In most situations, traditional electrospinning uses a solid metallic mandrel to collect the electrospun scaffolds. In contrast, the method and systems described herein employ a hollow mandrel with defined pores to allow pressurized air to be introduced within and expelled through the pores to create air jets that disrupt fiber deposition and prevent compaction of fiber deposition upon collection to form the non-woven scaffold. The exemplary perforated mandrel (FIGS. 6A and B) used to obtain the data presented in this Example was a 6.2 mm diameter stainless steel hollow tube (wall thickness 0.5 mm) with 750 micron pore diameters patterned with 2 mm spacing longitudinally and circumferentially with a 1 mm offset between rows circumferentially (Beverlin Manufacturing). This mandrel was outfitted at one end with an adapter to allow continuous rotation in the existing systems for even fiber collection over the mandrel. The opposite end was fitted with a one-way stopcock with a swivel male luer lock (Medex) to allow the introduction of pressurized air into the lumen of the mandrel while at the same time allowing continuous rotation.

PCL (120,000 MW) was electrospun from 1,1,1,3,3,3 hexafluoro-2-propanol (HFP) at a concentration of 150 mg/ml at standard processing conditions onto either the 6.2 mm inner diameter perforated stainless steel mandrel with either no airflow or airflow supplied at 100 kPa, or with a conventional solid stainless steel mandrel measuring 6.0 mm in outer diameter. The resulting random fiber orientation scaffolds were characterized with respect to scaffold morphology, structural properties through compliance, burst strength, and water permeability, using standard methods. Visual inspection showed that the scaffolds produced by this air disturbance method had an obvious increase in overall wall thickness (750 μm) compared to samples produced with zero air flow (350 μm) (when equal mandrel lengths and volumes of polymer solution were used). This demonstrates an increase in overall porosity when air flow was used. Nevertheless, the scaffolds were resistant to collapse, as measured by a Mitutoyo digital micrometer with a <1.5 Newton measuring force.

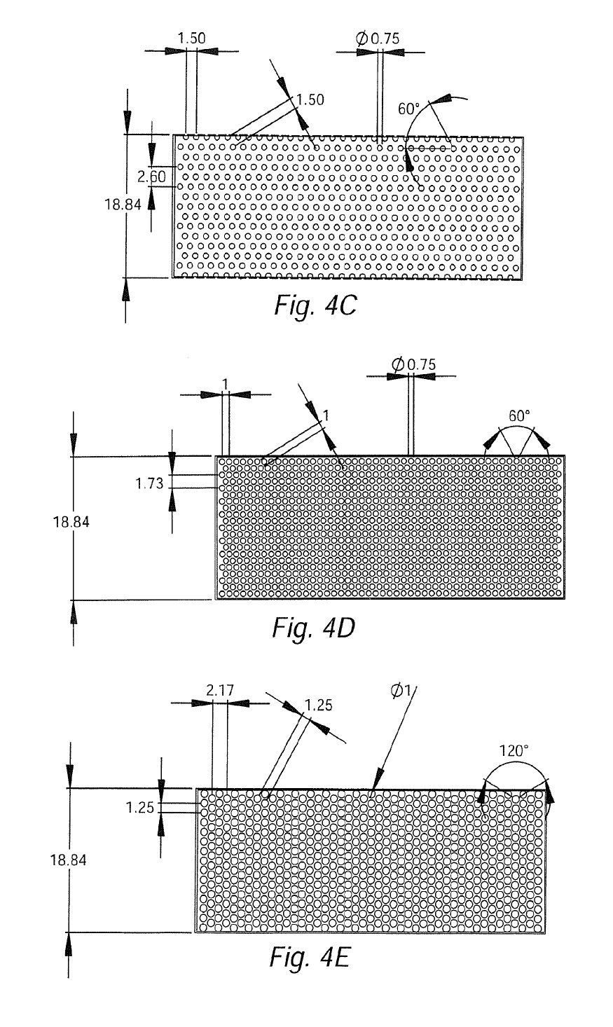

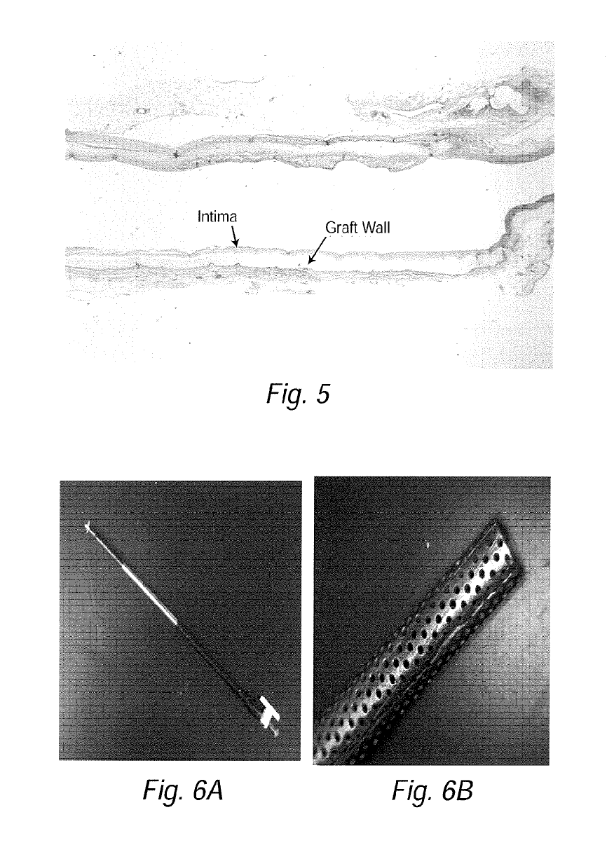

Upon examination of the structural micrographs, a clear difference in scaffold morphology was observed. As expected, the scaffolds fabricated on the solid mandrel had even, uniform surfaces (both internal and external) composed of very densely packed fibers (FIG. 7C and FIG. 8A). The surfaces of the scaffolds from the perforated mandrel when no air flow was applied were very similar except on the internal surface where the fiber density is less at sections over the open pores, compared to the areas of solid material (FIG. 7A). However, for the airflow samples, less dense fiber packing is seen on the external surface of the perforated areas with some raised regions (spikes) (FIG. 7B; FIG. 8B). This is in contrast to the zero airflow samples that resemble the solid mandrel, FIG. 7C.

A summary of the differences in scaffolds formed on perforated versus solid mandrels is presented in Table 1 for solid mandrel, zero airflow, and 100 kPa airflow (1.3×10⁻³ liters/second/pore) scaffolds fabricated (4 cm length with a constant volume of 1.2 ml electrospun for each, n=3). The mechanical testing (tensile testing, burst strength, and compliance) and whole graft water permeability methods used for characterization are standard tests [20].

TABLE 1
Summary of the results from the preliminary
physical characterization studies.

| | 0 kPa | 100 kPa | Solid |
|---|---|---|---|
| Mandel Type | Perforated | Perforated | Mandrel |
| Burst Strength (mm Hg) | 756 ± 31 | 769 ± 240 | 758 ± 168 |
| Compliance (%/100 mm Hg) | 1.2 ± 0.2 | 1.1 ± 0.1 | 0.6 ± 0.1 |
| Grafts Water Permeability | 53 ± 4 | 100 ± 5 | 49 ± 6 |
| (ml/cm² min) | | | |

The results of the water permeability study conducted at 120 mm Hg clearly demonstrated a more open pore structure with the permeability nearly doubling for the airflow mandrel with respect to the no airflow and solid mandrel. More importantly, this novel technique increased porosity without compromising the overall mechanical integrity based on the burst strength. In terms of compliance, the effects are small on these values but airflow electrospinning seems to allow the scaffolds to approach the values of soft tissues (e.g. artery) as compared to the solid mandrel scaffolds which provide more rigid structures.

In conclusion, these results demonstrate that air-flow impedance electrospinning is effective at creating a more porous structure without compromising mechanical integrity.

A cell seeding study was performed with immortalized endothelial cells to evaluate the scaffold's functional porosity. For static seeding, three ml of 1.5×10⁶ cells/ml were placed onto a 2×2 cm section of a scaffold produced by air-flow impedance or a solid mandrel scaffold then allowed to culture for 6 hours. For pressure seeding, 10 ml of 1.5×10⁶ cells/ml were forced manually (i.e. not using a controlled perfusion system) into the air-flow impedance scaffold contained on the perforated mandrel or a cannulated solid mandrel scaffold via a 10 ml syringe and then placed in media for 3 hours. The histology results showed that the static and pressurized seeding of the solid mandrel scaffolding resulted in a dense cellular layer on the luminal surface, and no cells were observed to have settled into the scaffolds. In contrast, the statically seeded airflow scaffold had cells infiltrating approximately half the scaffold thickness in regions over the pores and solely on the luminal surface otherwise. In marked contrast to these results, the constructs seeded by pressure seeding exhibited “plumes” of cells that were deeply imbedded throughout the cross section (thickness) of the scaffolds. The cells within these plumes were very uniformly distributed.

In summary, this data demonstrates the success of air-flow impedance electrospinning and the production of 3-D tissue engineering electrospun constructs.

### Example 2

Fabrication of air-flow impedance electrospinning mandrels to allow control over scaffold porosity by regulating airflow rate, pore diameter, and pore spacing as an examples for vascular graft development.

Previous electrospun scaffolds for various tissue engineering applications using solid mandrels have had limited success in regenerating tissues due to the lack of cellular infiltration due to tightly packed fibers. To overcome this limitation, novel perforated mandrels with pressurized airflow exiting the pores to impede fiber deposition have been developed, and are optimized resulting in the development of electrospun 3-D scaffolds with increased, controlled, porosity as compared to traditional electrospun scaffolds (solid mandrel). Significantly, these new methods of fabrication do not compromise the mechanical properties of the resulting scaffold.

The current electrospinning system utilizing a solid mandrel allows for mandrel rotation (0-5000 rpm) and oscillating translation (6 cm/s over a distance of 12 cm) permitting an even distribution of collected fibers on the mandrel (for configurations ranging from rectangular to tubular mandrels). Modification to one of the mandrel end-grips is necessary to accommodate the perforated mandrel and continuous pressurized air delivery while allowing rotation/translation for uniform scaffold fabrication. The end-grip to be modified is modular and requires minimal engineering and fabrication to allow exchange of solid and perforated mandrels under identical rotational and translation specifications.

Airflow Mandrel: As described in Example 1, data was obtained with a 6.2 mm diameter stainless steel hollow tube (wall thickness 0.5 to 0.75 mm) with 0.5 mm pore diameters patterned with 2 mm spacing longitudinally and circumferentially with 1 mm offset between rows circumferentially. Additional mandrels are fabricated with varying pore diameters and varying distances between pores, as well as various offsets, as required or desired for particular applications.

Scaffold Fabrication: To generate random fiber orientation scaffolds, a variety of synthetic polymers are utilized due to their varying mechanical properties and degradation rates. Exemplary polymers include poly(glycolic acid) (PGA) [19, 21], which is more rigid, crystalline, and rapidly degraded (<3 weeks) by hydrolysis; polydioxanone (PDO) [22] which is more elastic with a degradation time of 3-6 weeks, and PCL [7, 23] which is very elastic and slow degrading (6 mo. to a year) and mimics many of the properties of soft tissues. Using the novel airflow mandrels and electrospinning system described herein (<500 rpm mandrel rotation), the polymers are electrospun over a range of, for example, three polymer concentrations in HFP (approximately 70-200 mg/ml to create a minimum (˜100 nm), mid-range (˜700 nm), and maximum (˜1.5 μm) fiber diameter) and three applied air pressures (0, 50, and 100 kPa) to create non-woven scaffolding over a range of fiber diameters, mechanical properties, and porosities (zero applied pressure controls for electric field effects).

Scaffold Characterization: Standard protocols for measuring fiber diameter and porosity are used to characterize the scaffolds (internal and external surfaces, n=8) [22, 24]. The results are expressed as the average fiber diameter (nm), average pore area (μm²), and porosity (%) with standard deviation. For the perforated mandrel samples, the evaluation is conducted within the airflow regions (above pores) and the solid mandrel regions (between pores). Additionally, classical methods are used to determine the scaffold permeability (rate of water flow through a sample at a given hydrostatic pressure), effective pore area, and fiber diameter of hydrated fibrous structures [25] as well as the whole graft/scaffolding water permeability [20]. The results are reported as the average hydrated fiber diameter (nm), hydrated effective pore area (μm²), permeability (ml/cm² min), and standard deviation. Finally, the mechanical properties of the scaffolds (n=8), including the modulus, yield strength, and ultimate tensile strength are determined by uniaxial tensile testing and reported as the average modulus, yield strength, and ultimate tensile strength with standard deviation. Statistical analyses are used to confirm that the scaffolds exhibit increased porosity and mechanical properties comparable to scaffolds on a solid mandrel.

Results: Scaffolds with a wide range of possible porosities and mechanical properties are fabricated. These scaffolds display increased porosity without altering mechanical integrity. In addition, the water permeability for the air-flow impedance electrospun samples is greater than the solid mandrel and no airflow samples. These attributes maximize cellular infiltration and 3-D regenerative capacity of the scaffolds. In some embodiments, a perforated mandrel with a decreased pore spacing is used in order to create a more uniform is cell seeded (between pores) structure.

### Example 3

Cellular distribution and tissue development after static and pressurized cellular seeding of the scaffolds.

The use of air-flow impedance electrospinning provides increased scaffold porosity that allows enhanced cellular infiltration. This example describes direct comparisons of scaffolds statically seeded (i.e. in situ cellular integration of an acellular scaffold) with pressurized seeding (i.e. in vitro tissue engineering applications) to illustrate the overall advantages of the scaffolds of the invention, which are formed by airflow exiting the mandrel during fiber deposition, which increases scaffold porosity and enhances cellular infiltration after static and/or pressurized cell seeding.

Scaffold Preparation Scaffolds are disinfected in ethanol for 10 minutes followed by three rinses in sterile saline. For static cell seeding, the tubular scaffolds are cut longitudinally and opened to form a sheet that is used to create 10 mm diameter samples (10 mm biopsy punch) and placed in a 24-well culture plate for luminal surface seeding. For pressurized cell seeding, the tubular scaffolds are retained on the mandrel and disinfected. All scaffolds are rehydrated for one hour in DMEM/10% FBS at 37° C. prior to seeding.

Scaffold Cell Seeding: For static cell seeding, 10 mm scaffold samples are placed in 48-well tissue culture plates, a cloning ring is placed on the upper surface, and 1×10⁶ human human dermal fibroblasts are seeded and allowed to adhere and populate the scaffold. The fibroblasts are used to provide the large number of cells required while maintaining consistency over the course of the research (removes the large amount of variability associated with primary cell lines). For pressurized cell seeding, the mandrel containing the scaffolding or a 6 cm segment of the solid mandrel scaffold is cannulated to allow a cell seeding suspension (˜1×10⁶ fibroblasts/ml) to be infused via a syringe pump at a set, constant, metering rate/pressure (exact cell inoculation concentration and flow/pressure are constant for all scaffolds) through the scaffold structure. A pressure transducer is used in-line to measure and maintain a constant applied pressure. After seeding, the scaffolds are placed in a Petri dish for static culture. After 3 hours as well as 1, 7, and 21 days, the scaffolds (n=6 at each time point) are frozen for histologic assessment.

Cells infiltrate and migrate along the fibers composing the scaffold very rapidly. As an illustration, electrospun scaffolds were fabricated of dense highly aligned PCL fibers and seeded with human dermal fibroblasts on the surface of the fibers/scaffolding as well as the ends of the fibers (not shown). The results clearly demonstrated that the cells seeded on the scaffold surface had no infiltration into the scaffolding as expected after 7 days. Conversely, the cells seeded on the ends of the fibers had migrated >700 μm into the scaffolding. Similar results are seen for random fiber orientations [26]. Thus, once the cells have infiltrated, they migrate and provide an even cellular density throughout the scaffolding fairly rapidly.

Histological Evaluation: Cellular infiltration in terms of seeding depth as well as infiltration depth and density are determined, along with regenerative capacity based on collagen deposition. Sections of tubular scaffold are taken from the proximal, mid-graft, and distal region (reference point—cell suspension inlet). The cryosectioned samples are processed (sections taken at a minimum of 500 μm spacing) and stained with 4′,6-diamidino-2-phenyl-indole dihydrochloride (DAN) and a primary antibody for human collagen type I and examined by fluorescence microscopy with images obtained for quantification using ImageTool 3.0 software. The depth of cell infiltration is quantified by scanning across the sections and measuring the depth of penetration of all the deepest penetrating cells (dₘₐₓ) and normalizing to scaffold thickness (t) to determine the degree of cellular infiltration (DCI). The degree of ECM production infiltration is determined using the same general protocol as cell infiltration. The breadth of cellular and ECM production across the scaffold sections (gaps devoid of cells are expected between large pore spacing scaffolds) is determined by first dividing the maximum cellular infiltration depth area into quarters. At the depth levels of ¼, ½, and ¾ the maximum cellular infiltration, the distance between cells (d_{gap}) is determined and averaged. From this, the cellular distribution breadth is determined by normalizing to the pore spacing distance (dₚₒᵣₑ). Most importantly, the scaffold seeding effectiveness ratio (SSER) is calculated as the ratio of the degree cellular infiltration to cellular distribution breadth with a ratio of one representing a completely cellularized scaffold. The overall cellular density is quantified by dividing the graft cross-section into eight quadrants and counting the total number of cells present in each quadrant with the number of cells/unit area/quadrant as well as the percentage in each quadrant.

At zero airflow applied, cell infiltration is limited, but is increased over the solid mandrel scaffold due to local electric field effects (sharp edges of the pores). The air-flow impedance scaffolds exhibit enhanced cell infiltration throughout the scaffold thickness is (high density). Thus, the electrospun scaffolds of the invention are more conducive to 3-D tissue regeneration than are conventional scaffolds.

Design Modifications: The design of the mandrels and the conditions under which electrospinning is done are modified to create the optimum processing conditions for each scaffolding material to maximize the DCI and SSER (ideally approaching value of one) and 3-D regenerative capacity. Design modification of the mandrel to vary and optimize pore diameter and spacing between the pores (e.g. to reduce the necessity of cell migration between open pore zones and create a SSER approaching one) may be carried out. Such modifications may involve changes in the mandrel itself and/or changes in scaffold processing parameters as desired, to allow for the maximum DCI and optimum without sacrificing significant mechanical integrity.

### Example 4

### Tissue Engineering Applications

Scaffold fabrication techniques are developed which scale the mandrel to prepare scaffolds suitable for use in vascular tissue engineering, e.g. cylindrical scaffolds with a 2-4 mm internal diameter. The technique is also expanded to other mandrel configurations for use in engineering tissues such as bone and cartilage, e.g. either by in vitro cellular or acellular in situ applications.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

### REFERENCES

1. Mooney D, Langer R: Engineering biomaterials for tissue engineering: The 10-100 micron size scale. In: The Biomedical Engineering Handbook. Edited by Bronzino J. Boca Raton: CRC Press; 1995: 1609-1618.
2. Greisler H P, Gosselin C, Ren D, Kang S S, Kim D U: Biointeractive polymers and tissue engineered blood vessels. Biomaterials 1996, 17(3):329-336.
3. How T V, Guidoin R, Young S K: Engineering design of vascular prostheses. Proceedings of the Institution of Mechanical Engineers Part H, Journal of Engineering in Medicine 1992, 206(2):61-71.
4. Ramakrishna S, Fujihara K, Teo W E, Lim T C, Ma Z: Introduction to Electrospinning and Nanofibers: World Scientific Publishing Company, Incorporated; 2005.
5. Bowlin G L, Pawlowski K J, Stitzel J D, Boland E D, Simpson D G, Fenn J B, Wnek G E: Electrospinning of polymer scaffolds for tissue engineering. In: Tissue Engineering and Biodegradable Equivalents: Scientific and Clinical Applications. Edited by Lewandrowski K, Wise D, Trantolo D, Gresser J, Yaszemski M, Altobelli D. New York: Marcel Dekker, Inc.; 2002: 165-178.
6. Huang Z-M, Zhang Y-Z, Kotaki M, Ramakrishna S: A review on polymer nanofibers by electrospinning and their applications in nanocomposites. Composites Science and Technology 2003, 63(15):2223-2253.
7. Sell S A, McClure M J, Garg K, Wolfe P S, Bowlin G L: Electrospinning of collagen/biopolymers for regenerative medicine and cardiovascular tissue engineering. Adv Drug Deliv Rev 2009, 61(12):1007-1019.
8. Madurantakam P A, Cost C P, Simpson D G, Bowlin G L: Science of nanofibrous scaffold fabrication: strategies for next generation tissue-engineering scaffolds. Nanomedicine (Lond) 2009, 4(2):193-206.
9. Stankus J J, Guan J, Fujimoto K, Wagner W R: Microintegrating smooth muscle cells into a biodegradable, elastomeric fiber matrix. Biomaterials 2006, 27:735-744.
10. Li M, Mondrinos M J, Gandhi M R, Ko F K, Weiss A S, Lelkes P I: Electrospun protein fibers as matrices for tissue engineering. Biomaterials 2005, 26(30):5999-6008.
11. Barnes C P, Sell S A, Knapp D C, Walpoth B H, Brand D D, Bowlin G L: Preliminary investigation of electrospun collagen and polydioxanone for vascular tissue engineering applications. International Journal of Electrospun Nanofibers and Applications 2007, 1:73-87.
12. McManus M C, Boland E D, Koo H P, Barnes C P, Pawlowski K J, Wnek G E, Simpson D G, Bowlin G L: Mechanical properties of electrospun fibrinogen structures. Acta Biomater is 2006, 2:19-28.
13. McClure M J, Sell S A, Simpson D, Bowlin G L: Electrospun polydioxanone, elastin, and collagen vascular scaffolds: Uniaxial cyclic distension. Journal of Engineered Fibers and Fabrics 2009, 4(2):18-25.
14. Pham Q P, Sharma U, Mikos A G: Electrospun poly(epsilon-caprolactone) microfiber and multilayer nanofiber/microfiber scaffolds: characterization of scaffolds and measurement of cellular infiltration. Biomacromolecules 2006, 7(10):2796-2805.
15. Zhang Y, Ouyang H, Liim C T, Ramakrishna S, Huang Z-M: Electrospinning of gelatin fibers and gelatin/PCL composite fibrous scaffolds. Journal of Biomedical Materials Research Part B: Applied Biomaterials 2005, 72B:156-165.
16. Baker B, Gee A, Metter R, Nathan A, Marklein R, Burdick J, Mauck R: The potential to improve cell infiltration in composite fiber-aligned electrospun scaffolds by the selective removal of sacrificial fibers. Biomaterials 2008, 29:2348-2358.
17. Nam J, Huang Y, Agarwal S, Lannutti J: Improved cellular infiltration in electrospun fiber via engineered porosity. Tissue Eng 2007, 13(9):2249-2257.

We claim:

1. An electrospun material comprising regions of densely packed electrospun fibers which are not permeable to cells and porous regions which are permeable to cells.

2. An electrospun material comprising regions of densely packed electrospun fibers which are not permeable to cells and porous regions which are permeable to cells, wherein said electrospun material is formed by depositing incipient electrospun fibers onto a perforated mandrel while expelling a gas out of perforations in said perforated mandrel.

3. An artificial tissue or organ, comprising
electrospun scaffolding material comprising regions of densely packed electrospun fibers which are not permeable to cells and porous regions which are permeable to cells; and
a plurality of cells of interest associated with said electrospun scaffolding material.

4. The artificial tissue or organ of claim 3, wherein at least a portion of said plurality of cells of interest are capable of carrying out at least one function of a tissue or organ of interest.

5. The artificial tissue or organ of claim 3, wherein said plurality of cells of interest are comprised of a single type of cell.

6. The artificial tissue or organ of claim 3, wherein said plurality of cells of interest are comprised of more than one type of cell.

7. An artificial tissue or organ formed by
exposing electrospun material comprising regions of densely packed electrospun fibers which are not permeable to cells and porous regions which are permeable to cells to a plurality of cells of interest,
wherein said step of exposing is carried out in a manner that permits at least a portion of said plurality of cells of interest to infiltrate said electrospun material at said porous regions which are permeable to cells.

8. The artificial tissue or organ of claim 7, wherein said step of exposing is carried out in vitro.

9. The artificial tissue or organ of claim 7, wherein said step of exposing is carried out in vivo.

10. A mandrel for electrospinning fibers, comprising
a perforated support for receiving incipient electrospun fibers.

11. The mandrel of claim 10 wherein said perforations are arranged in a uniformly distributed pattern over said surface of said support.

12. The mandrel of claim 10 wherein said perforations are arranged in a non-uniformly is distributed pattern over said surface of said support.

13. A method for forming electrospun material comprising regions of densely packed electrospun fibers which are not permeable to cells and porous regions which are permeable to cells, said method comprising the step of
depositing incipient electrospun fibers on an outer surface of a perforated mandrel while directing a gaseous medium under pressure through perforations in said perforated mandrel toward said outer surface.

14. The method of claim 13, wherein said gaseous medium is air.

15. An electrospinning system, comprising:
a source for generating incipient electrospun fibers during an electrospinning process;
a perforated mandrel for receiving said incipient electrospun fibers during an electrospinning process; and
a gaseous medium pressure source for directing a gaseous medium under pressure through perforations in said perforated mandrel during an electrospinning process.

16. A method of in situ tissue regeneration, comprising
implanting into a subject in need thereof a scaffold comprising regions of densely packed electrospun fibers which are not permeable to cells and porous regions which are permeable to cells.

17. The method of claim 16, wherein said scaffold is formed by depositing incipient electrospun fibers onto a perforated mandrel while expelling a gas out of perforations in said perforated mandrel.
